(19) United States
(12) Patent Application Publication
(10) Pub. No.: US 2025/0029353 A1
(43) Pub. Date: Jan. 23, 2025

(54) SURGICAL OBJECT DETECTION MACHINE LEARNING

(71) Applicant: Nicholas Skapura, Warsaw, IN (US)

(72) Inventor: Nicholas Skapura, Warsaw, IN (US)

(21) Appl. No.: 18/776,555

(22) Filed: Jul. 18, 2024

Related U.S. Application Data
(60) Provisional application No. 63/527,641, filed on Jul. 19, 2023.

(51) Int. Cl. G06V 10/25; G06V 10/44; G16H 30/40

(52) U.S. Cl. CPC G06V 10/25; G06V 10/44; G16H 30/40; G06V 2201/07

(57) ABSTRACT

Systems and techniques may be used to perform surgical object detection machine learning. An example technique may include capturing a video of an operating room, the video including a set of frames, and generating, using an initial machine learning model, a set of bounding boxes corresponding to a specified object identified in one or more of the set of frames. The example technique may include receiving respective indications for each of the set of frames indicating whether each of the set of the frames were correctly labeled or incorrectly labeled for the specified object, and retraining the initial machine learning model using frames of the set of frames that were indicated to be incorrectly labeled for the specified object and corresponding bounding boxes of the set of bounding boxes to generate an updated machine learning model customized to the operating room. The updated machine learning model may be output.

### CLAIM OF PRIORITY

This application claims the benefit of U.S. Provisional Patent Application Ser. No. 63/527,641, filed on Jul. 19, 2023, the benefit of priority of which is claimed hereby, and which is incorporated by reference herein in its entirety.

### BACKGROUND

Orthopedic patient care may require surgical intervention, such as for lower extremities (a knee, a hip, etc.). For example, when pain becomes unbearable for a patient, surgery may be recommended. Many objects are used in a surgical field (e.g., an operating room), such as implants, a sterile drape, surgical equipment, cleaning equipment, etc. Tracking these objects is traditionally a manual process, taking significant time and resources, and prone to errors.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an operating room for training or using a machine learning model in accordance with at least one example of this disclosure.

FIG. 2 illustrates a block diagram of facilities for training or using a machine learning model to detect an object in accordance with at least one example of this disclosure.

FIG. 3 illustrates a user interface displaying an example output of a trained machine learning model in accordance with at least one example of this disclosure.

FIG. 4 illustrates a machine learning engine for surgical object detection in accordance with at least one example of this disclosure.

FIG. 5 illustrates a flowchart showing a technique for surgical object detection in accordance with at least one example of this disclosure.

FIG. 6 illustrates a block diagram of an example machine upon which any one or more of the techniques discussed herein may perform in accordance with at least one example of this disclosure.

### DETAILED DESCRIPTION

Systems and techniques described herein may be used for automatic surgical object detection. There are many objects in a surgical field or operating room during a surgical procedure that are beneficial to track. However, accurately detecting objects (including people, in some examples) is a challenging technical problem due to movement of objects, variance among different operating rooms, training time required for generating an accurate model, etc. The systems and techniques described herein provide a technological solution to these challenges by modifying a baseline model based on attributes of a current operating room. The baseline model may be initially trained at another operating room or at a previous time.

The baseline model may include an initial machine learning model, which may be trained to detect objects in a sterile field (e.g., an operating room). The initial machine learning model may be generated using a small seed model (e.g., with some labeled data). The initial machine learning model may be modified based on a supervised learning technique. For example, the initial machine learning model may generate a bounding box corresponding to a specified object. The specified object may be a particular object specified for a training (e.g., a surgical instrument, an implant, a patient, etc.). In some examples, a model may be trained successively or in parallel on different objects such that the model may detect more than one object. In other examples, a separate model may be trained for each object or each set of objects (e.g., a set of implants).

The initial machine learning model may output a label for whether or not an object is present in an image (e.g., within a bounding box). The label may be evaluated for whether it was correctly labeled by the initial machine learning model. The bounding box may be separately evaluated for whether it accurately bounds a detected object, in some examples regardless of whether the label was correct or not. The bound box may be accurate when it encompasses the object within a specified threshold. The initial machine learning model may be retrained using correct or incorrect label information or using correct or incorrect bounding box information. An updated machine learning model may be generated by the retraining. The updated machine learning model may be specific to attributes of the sterile field, such as objects present, lighting, arrangement, etc. The updated machine learning model may be used for one or more determinations before, during, or after a surgical procedure, such as determining whether a surgical drape is up, whether cleaning equipment is present, whether an anesthesia mask is on, or the like.

To retrain the initial machine learning model, video or images of the sterile field may be captured, for example using a camera. The camera may be moved around the sterile field in some examples. The video or images may be fed into the initial machine learning model, which may output a label or bounding box for a specified object. As the label or bounding box outputs are verified or discarded, a particular subspace of the sterile field may be identified where the initial machine learning model has worse outputs. For example, an area may be less well lit than the rest of the sterile field. The identified subspace may be captured with more video or images than other portions of the sterile field in order to obtain more data at the identified subspace. The identified subspace may include more labeled video frames or images than other portions of the subspace. In some examples, more than one subspace may be identified, for example when an area has a threshold percentage or number of labels or boundary boxes that are incorrect.

In some examples, boundary boxes that do not meet a threshold may be discarded. An expert may mark a label or bounding box as correct or incorrect. When an expert marks correct or incorrect for a bounding box, the expert may apply a score, such as on a scale of one to three, one to five, one to ten, etc. The scores may indicate a degree of precision in how well bounded an identified object is by the bounding box. Low scores (e.g., bottom 10%) may be discarded when updating the initial machine learning model.

The initial machine learning model may be retrained using a set of incorrectly labeled frames or images that have bounding boxes that are acceptable (e.g., bound the object within a threshold or range). These images and evaluation (e.g., incorrect label) may be fed forward into the initial machine learning model, which is retrained for a brief period of time (e.g., a half hour, an hour, a few hours, etc.). The period of time may correspond to a size of the model, for example a large model with over 3,000 images may use two or more hours, while a smaller model of 500 to 1,000 images may be limited to an hour or so. The timing may be approximate. The limited training time may be used to avoid overfitting an updated model to the retraining. The process (e.g., capturing video or images, evaluating labels or bounding boxes, retraining the model, etc.) may be iterated to further refine the updated model. As the iterations progress, the subspaces shrink and eventually disappear, which may be used as an indication that the updated model is sufficiently trained and ready for use.

After the updated model is generated, the updated model may be connected to a video feed in the sterile room to identify one or more objects as an output. The output may be directly provided to a user (e.g., displayed on a screen for a surgeon), or may be further processed, such as to determine whether a certain event has occurred, marking a surgery start or stop, an anesthesia start, stop, or interruption, or the like.

In some examples, the systems and techniques described herein may be applied to other fields than a surgical field. For example, other types of rooms may be used with the object recognition techniques described herein, such as a classroom, a house or apartment, a science lab, a physical therapy or rehabilitation clinic, other treatment facilities, or the like. By leveraging naïve models or models that are loosely similar to the target problem to be solved, a data labeling and annotation process that is typically performed manually is automated using the systems and techniques described herein.

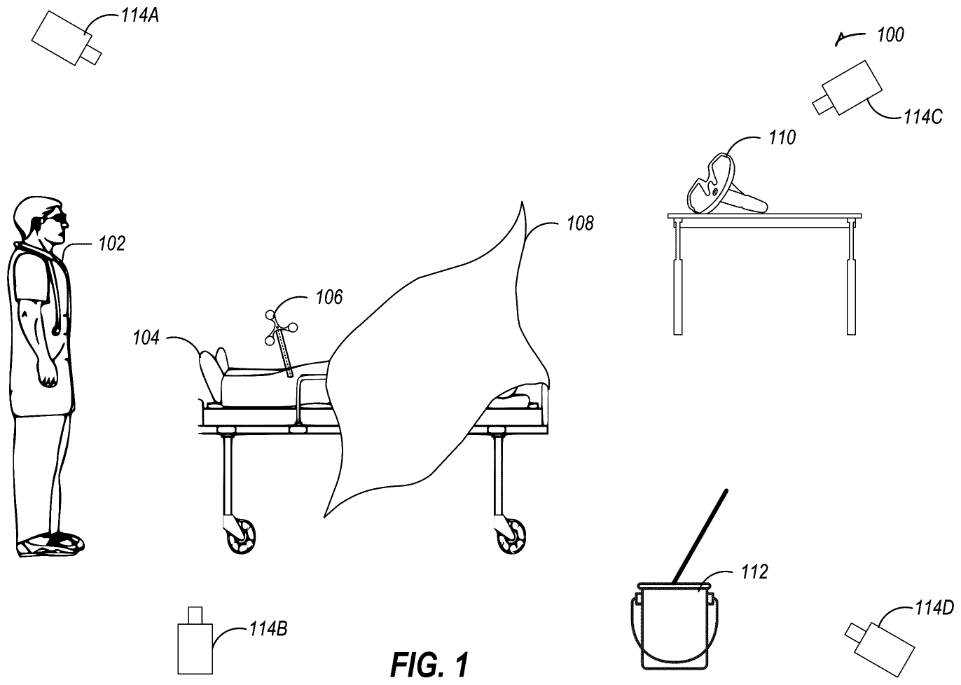

FIG. 1 illustrates an operating room 100 for training or using a machine learning model in accordance with at least one example of this disclosure. The operating room 100 includes a number of different objects that may be identified using the systems and techniques described herein. These objects may include a surgeon 102, a patient 104, a marker 106, a surgical drape 108, an implant 110 or other medical device, cleaning supplies 112, or the like. The objects may be captured by one or more cameras, such as a camera at one or more positions 114A, 114B, 114C, or 114D. Four camera positions are shown in FIG. 1, which may represent different locations that a single camera is moved to during a training phase. In some examples, more than one camera may be used. Training may occur while a camera is being moved, or only when the camera is stationary. The representative positions 114A, 114B, 114C, or 114D are examples to show different angles that may be used to capture the operating room 100, but other positions or fewer positions may be used.

After a camera captures a video, a set of frames of the video may be fed into a baseline trained model. The baseline trained model may be initially generated at another facility (e.g., not the operating room 100), or may be created as a simple seed model at the operating room 100 (e.g., using a limited number of labeled frames, such as 5, 10, 15, 20, 50, 100, etc.). When retraining the baseline trained model, a particular object may be selected, such as the surgeon 102, the patient 104, the marker 106, the surgical drape 108, the implant 110 or other medical device, the cleaning supplies 112, or the like. Using the implant 110 as an example, the baseline trained model may determine whether a particular frame includes the implant 110 or not, or a bounding box around an object. Even when the implant 110 is incorrectly labeled by the baseline trained model, the baseline trained model may output a bounding box. For example, the implant 110 may be in a frame captured by the camera at position 114C, and the baseline trained model may create a bounding box for the implant 110, but mislabel it as not being the implant 110. Even when the label is incorrect, the bounding box may be used (e.g., for retraining the baseline trained model). In some examples, such as when the camera is at position 114B, the implant 110 may not be present in a captured frame.

When the baseline trained model output is correct for a frame, the label and bounding box may be disregarded for retraining purposes because the training for that frame is complete. When model output is incorrect (e.g., the label is incorrect) for a frame, that frame may be stored with any one or more bounding boxes generated by the baseline trained model for the frame. The incorrectly labeled frame may be used in retraining the baseline trained model. The expected output of the model may be specified by a specialist or expert at the start of a segment of a video (e.g., “everything we are about to do in the video involves drape on”).

This process may be continued while the camera or objects in the operating room 100 are moved around. As objects are moved or manipulated or the camera moves, error subspaces (e.g., a mathematical or logical space derived from input space in a physical room) may be identified where the error rate is higher than other parts of the operating room 100 (e.g., the model may only fail when the drape is laying in a certain position). Identifying error subspaces may be used to generate an error map of the operating room 100 for the baseline trained model. A targeted sampling may be performed with video or images captured only from or more from the error subspaces where the model performs poorly.

The error subspace may include a portion of an input space. The input space may include each captured frame, as representing a point in space. The input space may include the space of all possible frames that may be captured by a camera. For example, an image of a table may be one point in space, an image of the table with a patient on it may be another point in space, etc.

Error subspaces may exist within the input space. An object at a certain angle may be considered in a top of a first frame, and the same object at the same angle in a bottom of a second frame may both be within the same error space (within the input space), even though they are in different parts of the room or frames. The image space may include a multidimensional data space where an image or a portion of an image may be a point in the space. In some examples, all images or frames that are captured may be used to define the input space, in other examples a subset of images or portions of images may be used. An error subspace may include a set of images or set of portions of images (e.g., belonging to an object) of the input space.

In some early iterations, bounding box quality may have poor examples. These poor examples may be removed or corrected through manual inspection. In later iterations, manual removal or correction may not be needed. In other examples, early iterations may be removed or corrected via an automated process. In an example, automated removal or correction may include clustering of bounding box coordinates, drawing a predicted bounding box around the cluster. In another example, automated removal or correction may include using a committee of homographies, where a homography is a mapping of one structure to a transformed version of the same structure. The committee of homographies may include randomly selecting a set of images and bounding boxes where the model had a correct output (which may optionally include images from another facility). For each new image, the set of mappings from the selected set of bounding boxes to the new image forms a committee of homographies (e.g., one homography for each of the selected images). The region in the new image with the most overlapping feature points from each homography may be determined. The region may be identified by finding where most of the homographies agree the object actually is. The boundary of this region may form the new bounding box. In still another example, a color histogram matching technique may be used for automated removal or correction. In this example, a histogram of pixel colors may be identified that is an average of the colors within the bounding boxes of a set of correct images. The model may output more than one possible bounding box for use in this example, for example where one of them is correct an incorrect one is selected by the model. The color histogram of all bounding boxes output by the model may be compared, and a best matching one may be automatically selected as the correct bounding box.

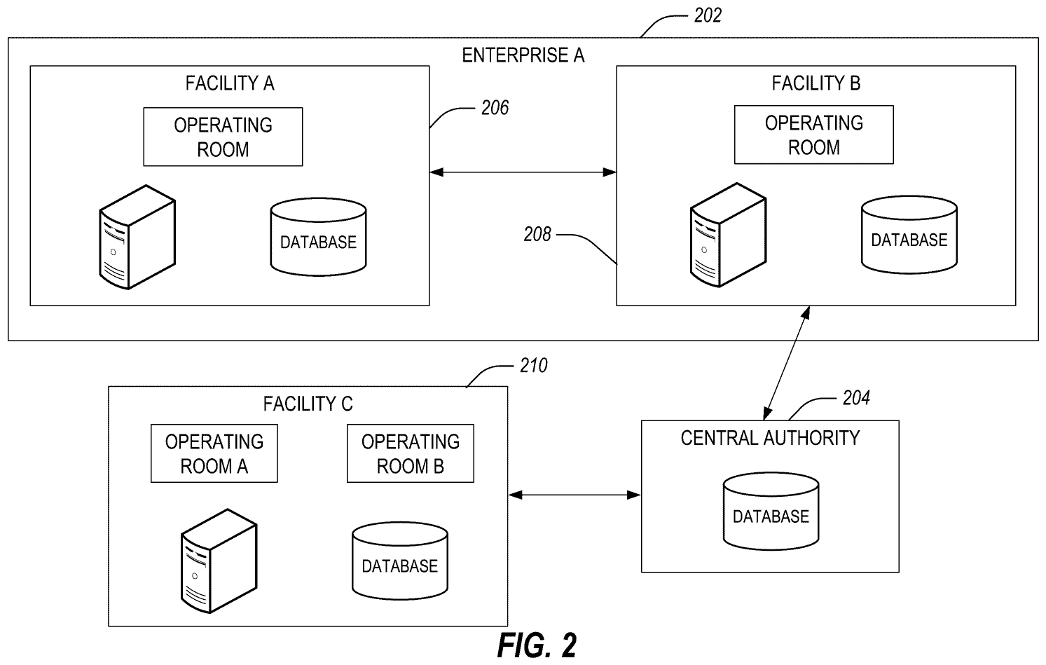

FIG. 2 illustrates a block diagram of facilities for training or using a machine learning model to detect an object in accordance with at least one example of this disclosure. FIG. 2 illustrates several facilities, such as facility A 206 and facility B 208, which are members of enterprise A 202, facility C 210, and a central authority 204, which may be associated with enterprise A 202 or may be independent. The central authority 204 may include a preferred or central facility of enterprise A 202, in some examples, where facilities A 206 and B 208 may be branch facilities. In other examples, the central authority 204 may include a governmental or independent governing or advisory body. The central authority 204 may provide a baseline model for use in retraining at a facility. For example, when the central authority 204 is associated with the enterprise A 202, the central authority 204 may store a baseline model in a database for use at facility A 206 or facility B 208. The baseline model may be generated at a preferred or central facility. When the central authority 204 is associated with a governmental, governing, or advisory body, the central authority 204 may provide a general baseline model to one or more facilities (e.g., facility C 210, which is not associated with enterprise A 202, and facility B 208, which is associated with enterprise A 202).

The association of facility A 206 or facility B 208 with enterprise A 202 may include an affiliation, a corporate or other business relationship, a contractual relationship, etc. In an example, a baseline model may be generated at facility A 206 and sent to facility B 208 for customization at an operating room of facility B 208. In other examples, the central authority 204 or an enterprise facility may be used to send a baseline model to both facility A 206 and facility B 208. In an example, the central authority 204 sends a baseline model to facility B 208, which then sends an optionally modified baseline model to facility A 206. Modifications to the baseline model may be made at facility B 208 before sending to facility A 206, such as to include one or more enterprise-specific modifications to the baseline model (e.g., enterprise branding, user interface, incorporation into an enterprise application or service, etc.).

Facility C 210 may include two or more operating rooms, such as operating room A and operating room B. Facility C 210 may retrain a baseline model (e.g., generated by the central authority 204) for each of the operating rooms A and B. The retraining may be specific to the operating room A or B (e.g., a first updated model is generated for operating room A and a second updated model is generated for operating room B). In some examples, the retraining may be generic to both operating rooms A and B (e.g., one updated model that is used in both), such as when the operating rooms have similar setups. In order to use the same updated model, a first iteration of retraining may be performed at both operating rooms (e.g., an operating room A iterated model and an operating room B iterated model). Outputs from the first iteration may be compared between the two iterated models, and when there is sufficient agreement, a single model may be used (e.g., stop training one of the models and iterate on the other model until there is a complete updated model).

In some examples, a facility (e.g., A 206, B 208, or C 210) may send information back to where the baseline model was generated (e.g., A 206, B 208, C 210, the central authority 204, or the like). The information sent back may be used to update the baseline model. For example, for enterprise A 202, when both facilities A 206 and B 208 have similar or the same issues in output label or bounding box, a baseline model for the enterprise A 202 may be modified to correct for the issues (e.g., for use in future operating rooms, another enterprise-related facility, etc.).

A baseline model may be generated at a facility that also updates the baseline model. For example, a baseline model may be generated at the operating room of facility A 206, such as using a seed model (e.g., a small number of expert-labeled images, such as 10, 100, etc.). Facility A 206 may use the seed model as a baseline model, and modify the seed model using the systems and techniques described herein to obtain an updated model for use at facility A 206.

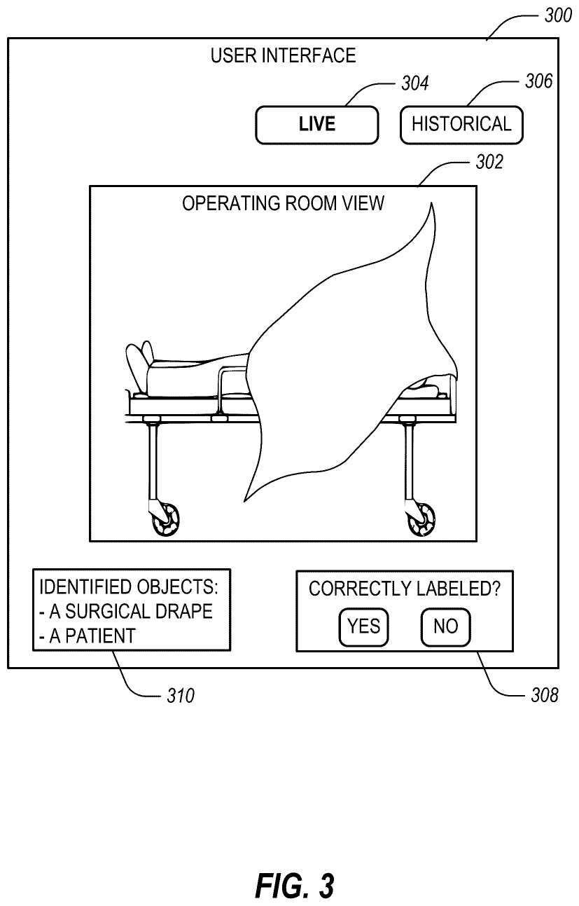

FIG. 3 illustrates a user interface 300 displaying an example output of a trained machine learning model in accordance with at least one example of this disclosure. The user interface 300 shown in FIG. 3 is an example, and includes optional components that may be omitted or supplemented (or both) without deviating from the disclosures herein. The user interface 300 shows example components, such as a video feed 302 of an operating room. In some examples, the video feed 302 may be replaced or supplemented by an image. The user interface 300 provides example identified objects 310, including a surgical drape and a patient identified from the video feed 302, such as at a current frame.

The user interface 300 may be used to view live 304 or historical 306 content. The user interface 300 may be used in an operating room, or for remote viewing of an operating room, which may include live 304 or historical 306 data. The live 304 view may include an output from a machine learning model for a current or recent frame of the video feed 302. In some examples, an output from the model may include the identified objects 310, a current surgical step (e.g., based on position or existence of an object in the video feed 302 or historical information), a safety status (e.g., anesthesia mask not currently on patient), or the like.

The user interface 300 may include a feedback component 308, which may be used by an expert to evaluate the output of the model (e.g., identified objects 310). The feedback component 308 may accept a determination of whether one or more objects from the video feed 302 were correctly labeled in the identified objects 310. In some examples, a bounding box may be displayed as an overlay on the video feed 302. In these examples, the feedback component 308 may include asking for a determination of whether the bounding box is acceptable, a value for the bounding box (e.g., from 1-5 where 1 is unacceptable and 5 is great or no improvement is needed), or the like.

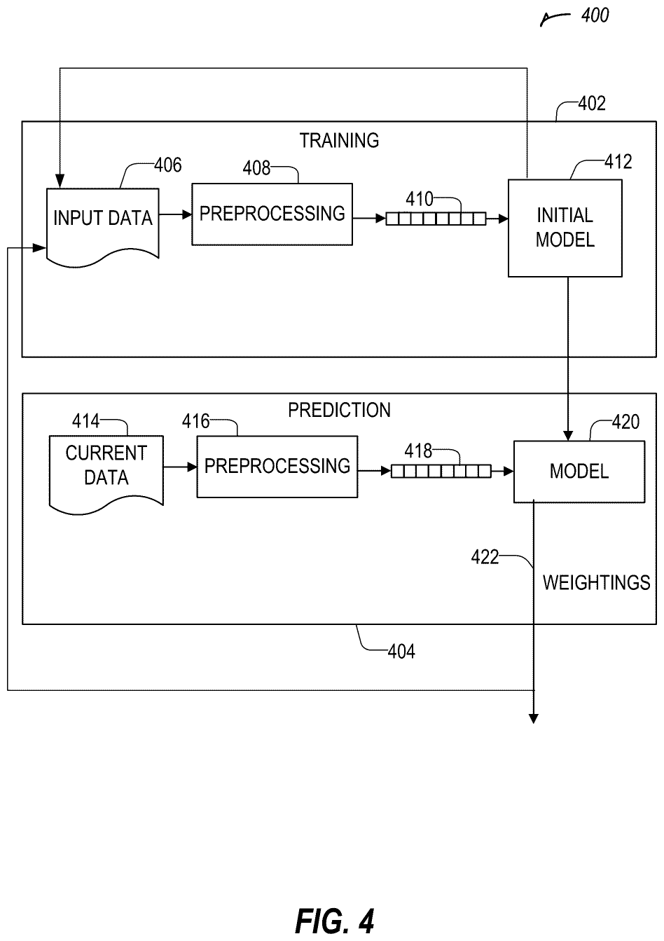

FIG. 4 illustrates a machine learning engine for surgical object detection in accordance with some embodiments. The machine learning engine can be employed within a mobile device (e.g., a cell phone) or a computer. A system may calculate one or more weightings for criteria based upon one or more machine learning algorithms. FIG. 4 shows an example machine learning engine 400 according to some examples of the present disclosure.

Machine learning engine 400 utilizes a training engine 402 and a prediction engine 404. Training engine 402 uses input data 406, after undergoing preprocessing 408, to determine one or more features 410. The one or more features 410 may be used to generate an initial model 412, which may be updated iteratively or with future unlabeled data.

The input data 406 may include one or more images or one or more frames of a video captured in a sterile environment (e.g., an operating room). In some examples, an object may be indicated, an object orientation or situation may be indicated (e.g., a location of a drape), or a surgical step may be used in the input data 406. In the prediction engine 404, current data 414 may be input to preprocessing 416. In some examples, preprocessing 416 and preprocessing 408 are the same. The prediction engine 404 produces feature vector 418 from the preprocessed current data, which is input into the model 420 to generate one or more criteria weightings 422. The criteria weightings 422 may be used to output a prediction, as discussed further below.

The training engine 402 may operate in an offline manner to train the model 420 (e.g., on a server). The prediction engine 404 may be designed to operate in an online manner (e.g., in real-time, at a mobile device, etc.). In other examples, the training engine 402 may operate in an online manner (e.g., at a mobile device). In some examples, the model 420 may be periodically updated via additional training (e.g., via updated input data 406 or based on unlabeled data output in the weightings 422) or user feedback (e.g., manual object labeling). The initial model 412 may be updated using further input data 406 until a satisfactory model 420 is generated. The model 420 generation may be stopped according to user input (e.g., after sufficient input data is used, such as 1,000, 10,000, 100,000 data points, etc.) or when data converges (e.g., similar inputs produce similar outputs).

The specific machine learning algorithm used for the training engine 402 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 402. In an example embodiment, a regression model is used and the model 420 is a vector of coefficients corresponding to a learned importance for each of the features in the vector of features 410, 418.

A label for the input data for the model 420 may include an identification of a particular object or a bounding box. Once trained, the model 420 may output an indication of whether a specified object is found, an identification of one or more objects, or a bounding box for an object in an image or frame of a video.

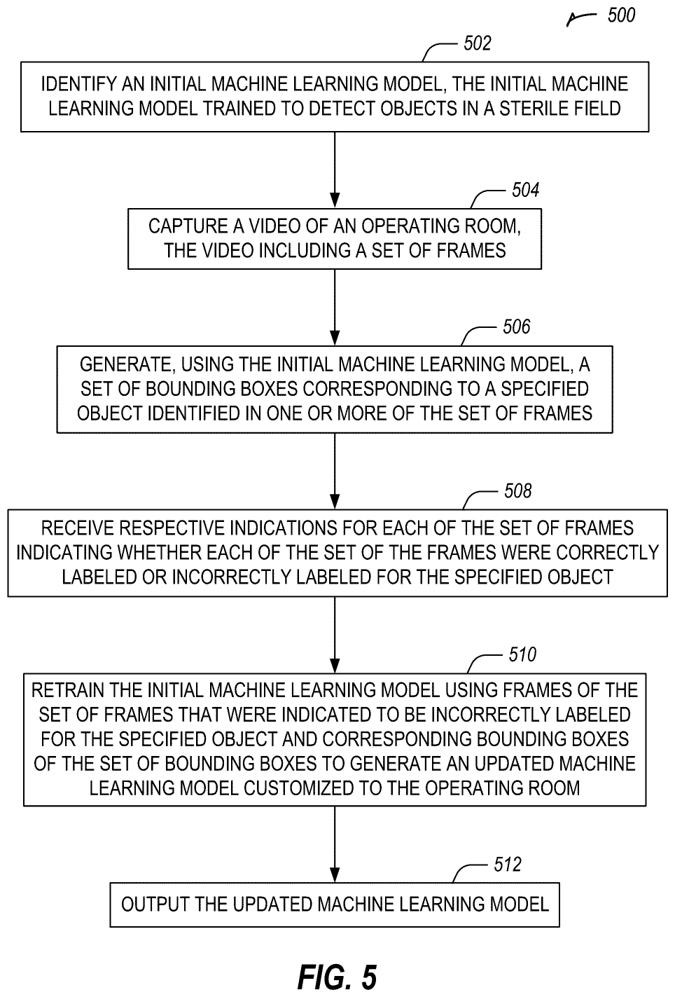

FIG. 5 illustrates a flowchart showing a technique 500 for surgical object detection in accordance with at least one example of this disclosure. The technique 500 may be performed by processing circuitry of a device, such as a robotic surgical device, a wearable device, a mobile device (e.g., a phone, a tablet, etc.), a computer (e.g., a laptop, a desktop, a server, etc.), or the like, locally, on an edge device, or in the cloud.

The technique 500 includes an operation 502 to identify an initial machine learning model, the initial machine learning model trained to detect objects in a sterile field. Operation 502 may include identifying the initial machine learning model trained at a second operating room. In another example, the initial machine learning model may be trained using a seed of images at the operating room or using simulated data.

The technique 500 includes an operation 504 to capture a video of an operating room, the video including a set of frames. The video may be captured while a camera is moved around the operating room (e.g., by a person, using an automated system, etc.).

The technique 500 includes an operation 506 to generate, using the initial machine learning model, a set of bounding boxes corresponding to a specified object identified in one or more of the set of frames. The specified object may include at least one of a surgical drape, cleaning equipment, an anesthesia mask, patient, a glove, a surgical device, or the like.

The technique 500 includes an operation 508 to receive respective indications for each of the set of frames indicating whether each of the set of the frames were correctly labeled or incorrectly labeled for the specified object. The respective indications may be received from a user (e.g., at a user interface), or automatically determined using a classifier or other technique.

The technique 500 includes an operation 510 to retrain the initial machine learning model using frames of the set of frames that were indicated to be incorrectly labeled for the specified object and corresponding bounding boxes of the set of bounding boxes to generate an updated machine learning model customized to the operating room. Operation 510 may include retraining the initial machine learning model for a limited time period to prevent overfitting (e.g., an hour).

The technique 500 includes an operation 512 to output the updated machine learning model (e.g., stored). In some examples, the technique 500 or portions of the technique 500 may be iterated (e.g., using the updated machine learning model as a new initial model) for example to generate a second updated machine learning model. The technique 500 may include retraining the updated machine learning model using a second specified object and the video or a second video.

The technique 500 may include generating an error mapping of the operating room for the initial machine learning model, the error mapping representing a subspace of an image space corresponding to portions of the operating room where the set of frames were incorrectly labeled. In this example, a second video of the subspace may be captured, the second video including a second set of frames. Using the initial machine learning model a second set of bounding boxes corresponding to the specified object identified in one or more of the second set of frames may be generated. This retraining may include using the second set of bounding boxes. The second set of bounding boxes may be output (e.g., for display).

The technique 500 may include selecting frames of the set of frames that were indicated to be correctly labeled for the specified object and corresponding bounding boxes of the set of bounding boxes. In this example, a set of mappings from the corresponding bounding boxes to a new image captured of the operating room may be generated. The technique 500 may include identifying a region in the new image having overlapping feature points from each mapping of the set of mappings. A boundary of the region may be used to form a new bounding box. The new bounding box may be output (e.g., for display).

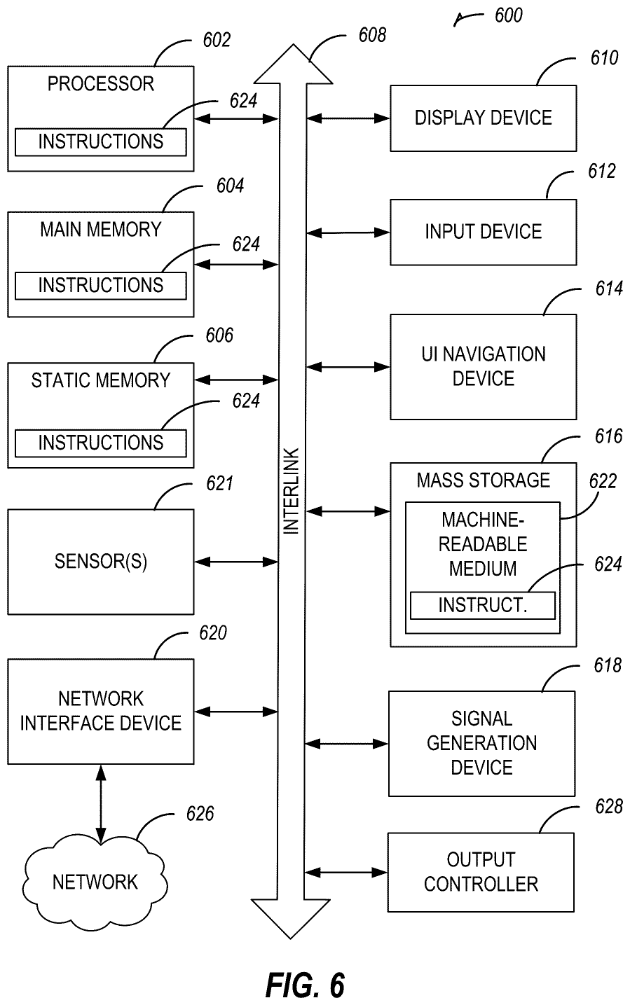

FIG. 6 illustrates a block diagram of an example machine 600 upon which any one or more of the techniques discussed herein may perform in accordance with some embodiments. This example machine can operate some or all of the Orthopedic Intelligence System discussed herein. In some examples, the Orthopedic Intelligence System can operate on the example machine 600. In other examples, the example machine 600 is merely one of many such machines utilized to operate the Orthopedic Intelligence System. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Each of the following non-limiting examples may stand on its own, or may be combined in various permutations or combinations with one or more of the other examples.

Example 1 is a method comprising: identifying an initial machine learning model, the initial machine learning model trained to detect objects in a sterile field; capturing a video of an operating room, the video including a set of frames; using the initial machine learning model, generating a set of bounding boxes corresponding to a specified object identified in one or more of the set of frames; receiving respective indications for each of the set of frames indicating whether each of the set of the frames were correctly labeled or incorrectly labeled for the specified object; retraining the initial machine learning model using frames of the set of frames that were indicated to be incorrectly labeled for the specified object and corresponding bounding boxes of the set of bounding boxes to generate an updated machine learning model customized to the operating room; and outputting the updated machine learning model.

In Example 2, the subject matter of Example 1 includes, generating an error mapping of the operating room for the initial machine learning model, the error mapping representing a subspace of the operating room where the set of frames were incorrectly labeled; capturing a second video of the subspace, the second video including a second set of frames; using the initial machine learning model, generating a second set of bounding boxes corresponding to the specified object identified in one or more of the second set of frames; and wherein retraining the initial machine learning model includes using the second set of bounding boxes.

In Example 3, the subject matter of Examples 1-2 includes, wherein identifying the initial machine learning model includes, identifying the initial machine learning model trained at a second operating room.

In Example 4, the subject matter of Examples 1-3 includes, training the initial machine learning model using a seed of images at the operating room.

In Example 5, the subject matter of Examples 1-4 includes, training the initial machine learning model using simulated data.

In Example 6, the subject matter of Examples 1-5 includes, retraining the updated machine learning model using a second specified object and a second video.

In Example 7, the subject matter of Examples 1-6 includes, wherein the method is iterated, and wherein on each iteration, generating bounding boxes includes generating a smaller set of bounding boxes and identifying a smaller error mapped portion of the operating room.

In Example 8, the subject matter of Example 7 includes, monitoring performance of the machine learning trained model from an iteration to detect when a video feed includes a mapped error space, and in response capturing a targeted sampling of frames within the mapped error space.

In Example 9, the subject matter of Examples 1-8 includes, wherein retraining the initial machine learning model includes retraining the initial machine learning model for a limited time period to prevent overfitting.

In Example 10, the subject matter of Examples 1-9 includes, selecting frames of the set of frames that were indicated to be correctly labeled for the specified object and corresponding bounding boxes of the set of bounding boxes; generating a set of mappings from the corresponding bounding boxes to a new image captured of the operating room; identifying a region in the new image having overlapping feature points from each mapping of the set of mappings; and using a boundary of the region to form a new bounding box.

Example 11 is at least one machine-readable medium, including instructions for operation at a mobile device, which when executed, cause processing circuitry to perform operations to: identify an initial machine learning model, the initial machine learning model trained to detect objects in a sterile field; capture a video of an operating room, the video including a set of frames; generate, using the initial machine learning model, a set of bounding boxes corresponding to a specified object identified in one or more of the set of frames; receive respective indications for each of the set of frames indicating whether each of the set of the frames were correctly labeled or incorrectly labeled for the specified object; retrain the initial machine learning model using frames of the set of frames that were indicated to be incorrectly labeled for the specified object and corresponding bounding boxes of the set of bounding boxes to generate an updated machine learning model customized to the operating room; and output the updated machine learning model.

In Example 12, the subject matter of Example 11 includes, wherein the instructions further cause the processing circuitry to perform operations to: generate an error mapping of the operating room for the initial machine learning model, the error mapping representing a subspace of the operating room where the set of frames were incorrectly labeled; capture a second video of the subspace, the second video including a second set of frames; generate, using the initial machine learning model, a second set of bounding boxes corresponding to the specified object identified in one or more of the second set of frames; and wherein retraining the initial machine learning model includes using the second set of bounding boxes.

In Example 13, the subject matter of Examples 11-12 includes, wherein to identify the initial machine learning model, the instructions further cause the processing circuitry to identify the initial machine learning model trained at a second operating room.

In Example 14, the subject matter of Examples 11-13 includes, wherein the instructions further cause the processing circuitry to perform operations to train the initial machine learning model using a seed of images at the operating room.

In Example 15, the subject matter of Examples 11-14 includes, wherein the instructions further cause the processing circuitry to perform operations to train the initial machine learning model using simulated data.

In Example 16, the subject matter of Examples 11-15 includes, wherein the instructions further cause the processing circuitry to perform operations to retrain the updated machine learning model using a second specified object and a second video.

In Example 17, the subject matter of Examples 11-16 includes, wherein the specified object includes at least one of a surgical drape, cleaning equipment, an anesthesia mask, patient, a glove, or a surgical device.

In Example 18, the subject matter of Examples 11-17 includes, wherein to retrain the initial machine learning model, the instructions further cause the processing circuitry to retrain the initial machine learning model for a limited time period to prevent overfitting.

In Example 19, the subject matter of Examples 11-18 includes, wherein the instructions further cause the processing circuitry to perform operations to iterate the method on the updated machine learning model to generate a second updated machine learning model.

Example 20 is a device comprising: processing circuitry; and memory including instructions, which when executed by the processing circuitry, cause the processing circuitry to perform operations to: identify an initial machine learning model, the initial machine learning model trained to detect objects in a sterile field; capture a video of an operating room, the video including a set of frames; generate, using the initial machine learning model, a set of bounding boxes corresponding to a specified object identified in one or more of the set of frames; receive respective indications for each of the set of frames indicating whether each of the set of the frames were correctly labeled or incorrectly labeled for the specified object; retrain the initial machine learning model using frames of the set of frames that were indicated to be incorrectly labeled for the specified object and corresponding bounding boxes of the set of bounding boxes to generate an updated machine learning model customized to the operating room; and output the updated machine learning model.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A method comprising:
identifying an initial machine learning model, the initial machine learning model trained to detect objects in a sterile field;
capturing a video of an operating room, the video including a set of frames;
using the initial machine learning model, generating a set of bounding boxes corresponding to a specified object identified in one or more of the set of frames;
receiving respective indications for each frame of the set of frames indicating whether each frame of the set of frames were correctly labeled or incorrectly labeled for the specified object;
retraining the initial machine learning model using frames of the set of frames that were indicated to be incorrectly labeled for the specified object and corresponding bounding boxes of the set of bounding boxes to generate an updated machine learning model customized to the operating room; and
outputting the updated machine learning model.

2. The method of claim 1, further comprising:
generating an error mapping of the operating room for the initial machine learning model, the error mapping representing a subspace of a model input space derived from space within the operating room where the set of frames were incorrectly labeled;
capturing a second video of the subspace, the second video including a second set of frames; and
using the initial machine learning model, generating a second set of bounding boxes corresponding to the specified object identified in one or more of the second set of frames;
wherein retraining the initial machine learning model includes using the second set of bounding boxes.

3. The method of claim 1, wherein identifying the initial machine learning model includes identifying the initial machine learning model trained at a second operating room.

4. The method of claim 1, further comprising training the initial machine learning model using a seed of images at the operating room.

5. The method of claim 1, further comprising training the initial machine learning model using simulated data.

6. The method of claim 1, further comprising retraining the updated machine learning model using a second specified object and a second video.

7. The method of claim 1, wherein the method is iterated, and wherein on each iteration, generating bounding boxes includes generating a smaller set of bounding boxes and identifying a smaller error mapped portion of the operating room.

8. The method of claim 7, further comprising monitoring performance of the updated machine learning model from an iteration to detect when a video feed includes a mapped error space, and in response capturing a targeted sampling of frames within the mapped error space.

9. The method of claim 1, wherein retraining the initial machine learning model includes retraining the initial machine learning model for a limited time period to prevent overfitting.

10. The method of claim 1, further comprising:
selecting frames of the set of frames that were indicated to be correctly labeled for the specified object and corresponding bounding boxes of the set of bounding boxes;
generating a set of mappings from the corresponding bounding boxes to a new image captured of the operating room;
identifying a region in the new image having overlapping feature points from each mapping of the set of mappings; and
using a boundary of the region to form a new bounding box.

11. At least one non-transitory machine-readable medium, including instructions for operation at a mobile device, which when executed, cause processing circuitry to perform operations to:
identify an initial machine learning model, the initial machine learning model trained to detect objects in a sterile field;
capture a video of an operating room, the video including a set of frames;
generate, using the initial machine learning model, a set of bounding boxes corresponding to a specified object identified in one or more of the set of frames;
receive respective indications for each frame of the set of frames indicating whether each frame of the set of frames were correctly labeled or incorrectly labeled for the specified object;
retrain the initial machine learning model using frames of the set of frames that were indicated to be incorrectly labeled for the specified object and corresponding bounding boxes of the set of bounding boxes to generate an updated machine learning model customized to the operating room; and
output the updated machine learning model.

12. The at least one non-transitory machine-readable medium of claim 11, wherein the instructions further cause the processing circuitry to perform operations to:
generate an error mapping of the operating room for the initial machine learning model, the error mapping representing a subspace of the operating room where the set of frames were incorrectly labeled;
capture a second video of the subspace, the second video including a second set of frames; and
generate, using the initial machine learning model, a second set of bounding boxes corresponding to the specified object identified in one or more of the second set of frames;
wherein retraining the initial machine learning model includes using the second set of bounding boxes.

13. The at least one non-transitory machine-readable medium of claim 11, wherein to identify the initial machine learning model, the instructions further cause the processing circuitry to identify the initial machine learning model trained at a second operating room.

14. The at least one non-transitory machine-readable medium of claim 11, wherein the instructions further cause the processing circuitry to perform operations to train the initial machine learning model using a seed of images at the operating room.

15. The at least one non-transitory machine-readable medium of claim 11, wherein the instructions further cause the processing circuitry to perform operations to train the initial machine learning model using simulated data.

16. The at least one non-transitory machine-readable medium of claim 11, wherein the instructions further cause the processing circuitry to perform operations to retrain the updated machine learning model using a second specified object and a second video.

17. The at least one non-transitory machine-readable medium of claim 11, wherein the specified object includes at least one of a surgical drape, cleaning equipment, an anesthesia mask, patient, a glove, or a surgical device.

18. The at least one non-transitory machine-readable medium of claim 11, wherein to retrain the initial machine learning model, the instructions further cause the processing circuitry to retrain the initial machine learning model for a limited time period to prevent overfitting.

19. A device comprising:
processing circuitry; and
memory including instructions, which when executed by the processing circuitry, cause the processing circuitry to perform operations to:
identify an initial machine learning model, the initial machine learning model trained to detect objects in a sterile field;
capture a video of an operating room, the video including a set of frames;
generate, using the initial machine learning model, a set of bounding boxes corresponding to a specified object identified in one or more of the set of frames;
receive respective indications for each frame of the set of frames indicating whether each frame of the set of frames were correctly labeled or incorrectly labeled for the specified object;
retrain the initial machine learning model using frames of the set of frames that were indicated to be incorrectly labeled for the specified object and corresponding bounding boxes of the set of bounding boxes to generate an updated machine learning model customized to the operating room; and
output the updated machine learning model.

20. The device of claim 19, wherein to identify the initial machine learning model, the instructions further cause the processing circuitry to identify the initial machine learning model trained at a second operating room.
